(19) United States
(12) Patent Application Publication
(10) Pub. No.: US 2005/0145523 A1
(43) Pub. Date: Jul. 7, 2005

(54) Visually coordinated absorbent product

(76) Inventors: Teresa M. Zander, Bondwl, WI (US); Kristi Jo Bryant, Appleton, WI (US); Debra A. Haase, Larsen, WI (US); Sheila M. Heyrman, Appleton, WI (US); Kim Hoertsch, Neenah, WI (US); Adrienne R. Loyd, Neenah, WI (US); William G. Reeves, Appleton, WI (US); Garry R. Woltman, Greenridge Drive, WI (US); Theodore T. Tower, Appleton, WI (US)

Correspondence Address: BRINKS HOFER GILSON & LIONE, P.O. BOX 10395, CHICAGO, IL, 60610, US

(21) Appl. No.: 10/749,871

(22) Filed: Dec. 30, 2003

(51) Int. Cl. A61L 15/00

(52) U.S. Cl. 206/438

(57) ABSTRACT

A visually coordinated absorbent product includes a product component having a body side liner, a garment side outer cover and an absorbent core disposed between the body side liner and the garment side outer cover. The product component has at least a first and second visual characteristic, wherein the first visual characteristic is different than the second visual characteristic. The product component is disposed in a packaging component having at least the first and second visual characteristics. In one embodiment, the packaging component is an individual pouch sized and configured to hold a single, individual product component. In various embodiments, the visual characteristics can include color, embossment, printing, and/or side sealing.

### BACKGROUND

The present invention relates generally to absorbent products, and in particular, to visually coordinated absorbent products.

Typically, absorbent products such as pads or panty liners include one or more elements, such as an outer cover and peel strip. Such products are often individually wrapped in a pouch or similar package, or are wrapped as a group of products. A plurality of products, whether or not individually wrapped, are also typically sold in bulk packaging, such as a bag or box. Often, the various elements of the product, the individual pouch and the bulk packaging are individually designed without any effort to coordinate the appearance or aesthetics of the various components. For example, a typical incontinent pad may be solid white or white with a blue stripe along the body side surface and have a peel strip with a print that is pink or blue. The pad is packaged in a solid white or solid pink pouch, with the plurality of pouches packaged in a bag perhaps having some blue or pink. In this way, while some colors (e.g., white or pink) are used on each of the components, they are not coordinated between the product and individual wrapper and/or bulk packaging. As such, the consumer is not made confident that the overall product, including the pad, the pouch and/or the packaging, was designed together. Accordingly, a need remains for an absorbent product that is visually coordinated with itself or with its packaging so as to invoke an impression from the user that the products were designed together and are of a high quality.

In addition, a consumer may not want others to know that they are carrying an absorbent product. A consumer may want the product to be masked or hid or otherwise disguised. Current pouches are typically made from a relatively heavy, uniform-micro embossed, uniform-colored film material which can prevent an observer from discerning what is contained in the pouch. Such materials can be relatively expensive and bulky. Lighter weight materials, however, are typically more see-through, thereby allowing observers to discern the contents of the pouch. Current pouches also appear rectangular in shape containing linear lines. The combination of the material and construction can give the wrapped product an appearance easily recognized by others as an absorbent product. Accordingly, a need remains for an absorbent product that is visually coordinated with itself and/or with its packaging, and which is not discernable through relatively see-through packaging and rectangular construction.

### SUMMARY

Briefly stated, in one aspect, a visually coordinated absorbent product includes a product component having a body-side liner, a garment-side outer cover and an absorbent core disposed between the body side-liner and the garment-side outer cover. The product component has at least a first and second visual characteristic, wherein the first visual characteristic is different than the second visual characteristic. The product component is disposed in a packaging component having at least the first and second visual characteristics. In one embodiment, the packaging component is an individual pouch sized and configured to hold a single, individual product component. In various embodiments, the visual characteristics can include color, embossment, printing, and/or side sealing.

In another aspect, a first packaging component has at least first and second visual characteristics and a product component disposed therein. A second packaging component also has the first and second visual characteristics. The first packaging component is disposed in the second packaging component.

In yet another aspect, a visually coordinated absorbent product includes a product component configured in a packaged configuration and having a first and second side defining an outer surface when configured in the packaged configuration. The product component includes a body side liner, a garment side outer cover and an absorbent core disposed between the body side liner and the garment side outer cover. The product component includes at least a first and second element defining the outer surface, wherein the at least first and second elements have a first and second color. The packaging component has first and second colors that are coordinated with the first and second colors of the product component. In one embodiment, at least one of the hue, saturation/vividness and luminosity are coordinated between the respective first and second colors of the product and packaging components respectively.

The various aspects provide significant advantages over other absorbent products. For example and without limitation, in the first aspect, the user is provided with an impression that the product is of high quality, and that the product and packaging were designed together, rather than piece meal. In addition, the visually coordinated product and packaging provides the user with an emotional benefit, allowing them to feel more feminine with respect to female absorbent products, and to feel better about the product they are using. The visual characteristics can also identify the product for the user, for example provide source identification.

With respect to the other aspects, the coordination of the elements of the absorbent products, or the coordination of the absorbent product with the packaging, allows the manufacturer to use a relatively see-through packaging material without sacrificing the ability to hide the contents of the packaging. For example, a relatively light basis weight non-woven material can be used for the packaging, thereby reducing the overall costs of the product.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

FIG. 1 is perspective view of a first embodiment of an individual packaging component.

FIG. 2 is a perspective view of a second embodiment of an individual packaging component.

FIG. 3 is a perspective view of an individual packaging component in an open configuration with an absorbent product component in a partially unfolded configuration.

FIG. 4 is a perspective view of a packaging component configured to hold a plurality of product components.

FIG. 5 is a perspective view of a product component in a partially folded configuration.

FIG. 6 is a body-side plan view of an exemplary product component with portion thereof partially cut away.

FIG. 7 is a perspective view of one embodiment of a product component with a packaging component.

FIG. 8 is a perspective view of another embodiment of a product component with a packaging component.

FIG. 9 is one embodiment of a peel strip having a pattern printed thereon.

FIG. 10 is a portion of one embodiment of a packaging component having a pattern printed thereon.

FIG. 11 is another embodiment of a peel strip having a pattern printed thereon.

FIG. 12 is a portion of another embodiment of a packaging component having a pattern printed thereon.

FIG. 13 is a schematic graphical illustration of color hue, luminosity and saturation/vividness.

### DETAILED DESCRIPTION OF THE PRESENTLY PREFERRED EMBODIMENTS

The term “bodyside” should not be interpreted to mean in contact with the body of the user, but rather simply means the side that would face toward the body of the user, regardless of whether an undergarment is actually being worn by the user and regardless of whether there are or may be intervening layers between the component and the body of the user. Likewise, the term “garment side” should not be interpreted to mean in contact with the garments of the user, but rather simply means the side that faces away from the body of the user, and therefore toward any outer garments that may be worn by the user, regardless of whether the undergarment is actually being worn by a user, regardless of whether any such outer garments are actually worn and regardless of whether there may be intervening layers between the component and any outer garment.

The phrases “removeably attached,” “removeably attaching,” “removeably connected,” “removeably engaged,” “releasably attached,” “releasably connected,” or “releasably engaged,” and variations thereof, refers to two or more elements being connected or connectable such that the elements tend to remain connected absent a separation force applied to one, both or all of the elements, and where the elements are capable of being separated upon the application of a separation force. The required separation force is typically beyond that encountered while wearing the absorbent garment.

The phrases “fixedly secured,” “fixedly engaged,” “fixedly attached,” “fixedly connected,” and variations thereof, refers to two or more elements being connected or connectable such that they are not disconnected or otherwise separated, and are not intended to be separated or disconnected by the end user, during the normal operation and use of the absorbent garment. Moreover, the separation of two elements being so connected is likely to damage or make unusable at least one of the two elements.

The terms “connecting,” “coupled,” “attached,” and “secured,” and variations thereof, broadly covers two or more items being directly connected one to the other, or by way of one or more intervening members or components.

It should be understood that the term “personal care product” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, personal care products, as used herein, includes without limitation diapers, pull-up garments, adult incontinence garments, male incontinence products, tampons, vaginal suppositories, panty liners, pads, sanitary napkins, tissues, wipes, etc. For example, personal care products include without limitation Poise® feminine care products, including pantiliners and pads, and Kotex® feminine care products, including pads, tampons and liners, all available from Kimberly-Clark Corporation, Neenah, Wis. Various exemplary products are disclosed in U.S. Pat. No. 6,315,765, entitled “Elasticized Absorbent Pad,” and U.S. patent application Ser. No. 10/392,116, filed Mar. 19, 2003 and entitled “Multilayer Absorbent Article”, the entire disclosures of which are hereby incorporated herein by reference.

Referring to FIGS. 3, 6 and 7, an absorbent product 10 is shown as including various elements, i.e., an outer cover 46 (otherwise referred to as a baffle), an absorbent core 48, a tissue layer 6, a surge layer 4 and a body side liner 44. Preferably, the body-side liner 44 is liquid-permeable. In one embodiment, portions of the surge layer, tissue layer and absorbent core are visible to the user through the body-side liner, which is at least partially see-through. The absorbent product 10 also has a first side and a second side 18. The first and second sides 16, 18, respectively, are the longitudinal sides of the elongated absorbent product. The sides can be contoured, for example in a concave shape, or they can be linear. The sides can further include flaps (not shown) that extend laterally outward. In one embodiment (not shown), one or more elastic elements are disposed along the sides to form a gasket with the body of the user. In one embodiment, the elastic elements are disposed between the liner and the outer cover.

The absorbent product 10 has a first body side surface 20 and a second garment side surface 22. Applied to at least a portion of the second garment side surface 22 is a garment attachment adhesive. In various embodiments, the garment attachment adhesive is configured as a single band of adhesive or as two or more spaced apart strips. Alternatively, the garment attachment adhesive includes a swirl pattern of adhesive which encompasses a major portion of the second garment surface 22 of the absorbent article 10.

Another element if the product is a release strip 28, also known as a releasable peel strip, which is removably secured to the garment attachment adhesive and serves to prevent premature contamination of the adhesive before the absorbent article 10 is secured to the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIG. 6, or it may have a lesser length than the outer cover, as shown in FIG. 5.

The body side liner 44, which is preferably liquid permeable, is formed from one or more of a nonwoven material such as spunbond or a perforated thermoplastic film. The term “non-woven” web or material, as used herein, means a web having a structure of individual fibers or filaments that are interlaid, but not in an identifiable manner and without the aid of textile weaving or knitting, as in a knitted or woven fabric. The baffle 46 should be liquid-impermeable and can be formed out of a thin layer of thermoplastic film, such as polyethylene. The liquid permeable liner 44 and the liquid-impermeable baffle 46 are peripherally sealed together to enclose the absorbent core 48 to form the absorbent article 10. Alternatively, the liner 44 can be wrapped around both the absorbent 48 and the baffle 46 to form a wrapped pad. The liner 44 and baffle 46, and other components of the absorbent product, can be joined for example with adhesive bonds, sonic bonds, thermal bonds, pinning, stitching or any other attachment techniques known in the art, as well as combinations thereof.

The absorbent core 48 is designed to absorb body exudates, including menstrual fluid, blood, urine, and other body fluids. The absorbent core 48 can consist of one or more layers of absorbent material. The layers can consist of similar materials or different materials. Suitable materials for the absorbent core 48 include cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has great absorbency.

The absorbent core 48 can also be formed from a composite comprised of a hydrophilic material that can be formed from various natural or synthetic fibers, wood pulp fibers, regenerated cellulose or cotton fibers, or a blend of pulp and other fibers. A preferred material is an airlaid tissue.

In one embodiment, the absorbent core also includes a superabsorbent material, which increases the ability of the absorbent core to absorb a large amount of fluid in relation to its own weight. Typical superabsorbents used in absorbent articles such as sanitary napkins, can absorb anywhere from 5 to 60 times their weight in body fluid. The superabsorbent materials can be inserted as particles or in sheet form. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25. Other types of superabsorbent materials known to those skilled in the art can also be used.

Additional layers or substrates, including for example, the liquid acquisition and distribution layer 5, also referred to as a surge or transfer layer, and a tissue layer 6 are also incorporated into the absorbent product, for example between the liner 44 and the absorbent core 48. In one embodiment, the transfer layer is shorter than the absorbent core. In one embodiment, the absorbent core, transfer layer and other components, such as tissue layers, are free floating (unattached) between the outer cover and the liner, which are secured along only the peripheral edges thereof. Alternatively, the absorbent core, transfer layer and other components are attached to one or both of the outer cover and liner and/or to each other.

Referring to FIGS. 3, 5 and 6, the absorbent product 10 is shown in a folded configuration. For example, the absorbent product can be folded along a pair of fold lines 30, 32 to form a tri-fold configuration. In other embodiments, the absorbent product can be bi-folded, flat or rolled. The absorbent product is then inserted into an individual packaging component, otherwise referred to as a wrapper or pouch. Alternatively, a plurality (meaning two or more) of product components can be disposed in a single pouch, and a plurality of pouches can be disposed in a package (shown in FIG. 4). One product/packaging configuration is shown in U.S. Pat. No. 6,601,706, which is hereby incorporated by reference. The products can be oriented in various ways within the individual packaging component, for example with the fold lines 30, 32 running parallel or perpendicular to the sides of the packaging component 68, 70.

In one embodiment, the packaging component is formed as a portion of the product component. For example, an outer cover, such as the baffle material, can form a packaging component, as shown for example in U.S. Pat. No. 5,993,430, which is hereby incorporated herein by reference. It should be understood that in these embodiments, the product component is considered to be disposed in the packaging component when the packaging element is sealed or otherwise closed. In other embodiments, the product component is rolled, with a flap portion of a packaging component overlying a panel portion of the packaging component, regardless of whether the packaging component is integral with the product component or separate therefrom.

Referring to FIGS. 1, 3A-3F and 4, the pouch 50 is formed from a strip or web 52 of material having a first and second ends having free edge 54, 56. It should be understood that the term “free edge” refers to an edge that is unattached after the package component is opened, regardless of whether the free edge is attached when the package component is closed. Accordingly, one or both of the free edges may be formed along a perforation line, or may be adhered to an underlying layer, with the edge defined by the perforation line being a “free edge” after the perforation line is broken. The free edge can be a single layer cut or formed edge, or can include a double-layer folded edge, or can include an edge formed by a plurality of layers. The pouch material can be formed from a non-woven material, films, paper, laminates, and/or cloth (including woven) materials, and combinations thereof. For example, the pouch can be made as disclosed in U.S. patent application Ser. No. 10/022,808, filed Dec. 18, 2001, the entire disclosure of which is hereby incorporated herein by reference. In one embodiment, the pouch is made of a film/spunbond laminate material available from Kimberly-Clark Corp, and know as HBSTL (“highly breathable stretch thermal laminate”), and which material is further disclosed in U.S. Pat. No. 6,276,032, the entire disclosure of which is hereby incorporated herein by reference. Various embodiments of a non-woven pouch material can have a basis weight less than about 1.50 osy, alternatively less than about 1.0 osy, or alternatively between about 0.50 osy and about 0.60 osy.

Each of the first and second ends are folded along fold lines 58, 60 that define the top and bottom edge of the pouch respectively. The folded pouch has a back 62, a front 64 and a flap 66. The front and back 64, 62 are secured along side edges 68, 70 thereof to form a pocket shaped to receive the absorbent product. In one embodiment, the pocket and pouch are shaped and dimensioned to receive a single product component, which is individually wrapped in the pouch. The flap 66 is folded over the front 64 such that the free edge 54 of the flap overlies the front 64. The front 64 has a covered or overlap portion 57 extending between the free edge 54 (exterior) and the free edge 56 (interior), which covered or overlapped portion 57 underlies the flap. In one embodiment, there is no overlap. Rather, the free edges 54, 56 abut. In one example, the portion 57 has a length of about 5 mm between the edges 54, 56. In various embodiments, the overlap distance is less than or equal to about 95% of the overall packaging component length in a closed configuration, more desirable less than or equal to about 35% of the packaging component length, and more desirably less than or equal to about 20% of the packaging component length. In various embodiments, the free edge 54 is positioned a distance from either edge 58, 60 that is greater than or equal to about 10% of the overall length of the packaging component (in a closed configuration), more desirably greater than or equal to about 30%, and more desirably about 50% of the packaging component length. The front further includes an uncovered second portion 59 extending between the free edge 54 and the bottom edge 58. Of course, it should be understood that the length and width of the product and packaging components can vary according to the type of product and the size of the product.

In one embodiment, the flap 66 is releasably secured to the front 64. For example, a fastening element 72, shown as a tab in FIG. 3A, is secured across the free edge 54 of the flap 66 to secure the flap 66 to the front 64. The fastening element can be releasably secured to both of the flap and front, or it can be fixedly secured to one of the flap and front and releasably secured to the other thereof. The fastening element can be formed as adhesive (e.g., tape), a snap, a button, a mechanical fastener (e.g., hook and loop), a tie, or as any other device known by those skilled in the art. The adhesive can be applied as a ribbon, dot or swirl pattern. The fastening element can have various alternative shapes, including by not limited to a square, rectangle, triangle, circle, oval, obround, oblong or diamond shape, or any other irregular shape or pattern. In an alternative embodiment, the fastening element is formed on the inside of the flap such that it engages the front as the flap is folded thereover and is not visible to the user. In yet another alternative embodiment, the flap is simply sealed to the front with a heat seal or other weld, with the weld defining the fastening element. In another embodiment, the flap is not sealed or otherwise attached to the front, but rather is simply folded thereover. Alternatively, the sides of the flap are sealed to the back and to the front, with the side seals being breakable in response to a user grasping and lifting the flap.

In some embodiments, the flap 66 is refastenably secured to the front 64, while in others, the flap is not intended to be secured to the front once the packaging component is opened. For example, in one embodiment, the free edge 54 is defined by a perforation line, with the flap not being refastenable after the perforation is broken.

A pair of side seals 74 secures the front 64 to the back 62, and the flap 66 to the back 62 and to the front 64. The side seals are desirably formed after the front is folded over the back and the flap is folded over the back and the front, although the front could first be sealed to the back, and the flap then sealed to one or both of the back and front. In an alternative embodiment, the flap is not sealed at all along the sides thereof, or anywhere else. In one embodiment, the side seals 74 are frangible, meaning they can be easily broken such that the flap 66 can be separated from the front 64 and back 62, and such that the front 64 can be easily separated from the back 62, wherein the product component 10 is exposed for removal from the pouch by the user.

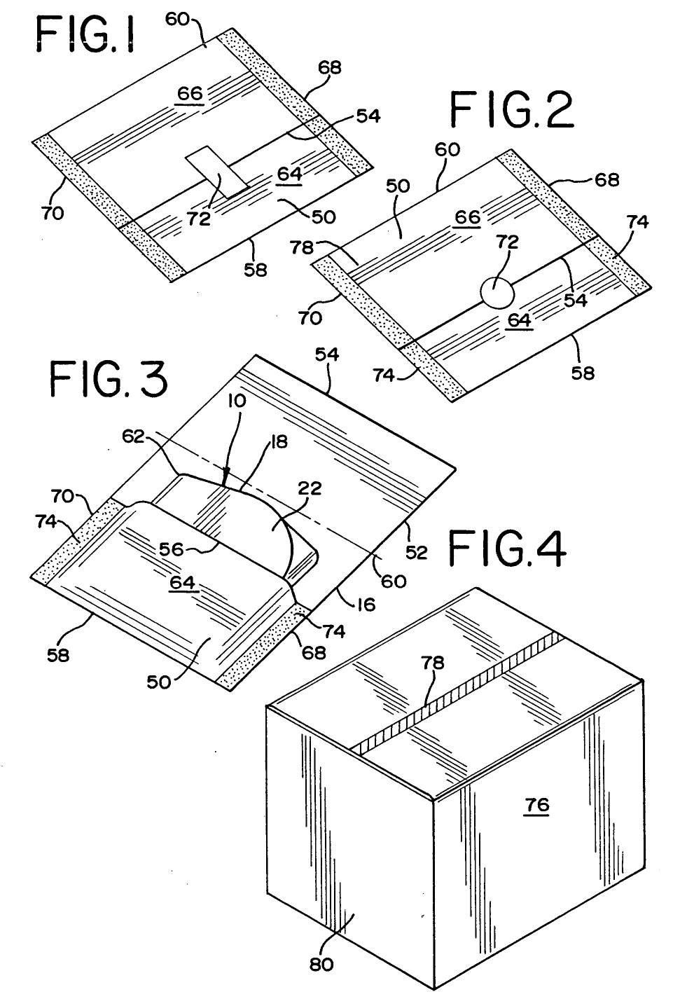

Referring to FIG. 4, a plurality of absorbent products 10, whether individually wrapped as shown in FIGS. 1-3, or left unwrapped, are packaged in a bulk packaging component 76, meaning a component capable of holding two or more absorbent products. In one embodiment, the packaging component is formed as a bag having at least one side seal 78 securing a pair of edges of the bag together. In other embodiments, the plurality of absorbent products are packaged in a box or carton.

Referring to FIGS. 1-3, the packaging component 50 has at least a first and second visual characteristic. The term “visual characteristic” means a feature or characteristic that is discernible by sight during the normal use of the component, and includes for example and without limitation color, shape, embossing, patterns (e.g. by printing), sealing patterns, etc. For example, the wrapper 52, preferably an outer surface 78 thereof including for example the back 62, flap 66 and the portion of the front 64 not covered by the flap, may have a first color and the outer surface of the fastening element 72 may have a second color, or the outer surface of the wrapper 52 may have a plurality of colors, including a first and second color. Alternatively, a portion or the entirety of the outer surface of the wrapper 52 may have an embossed pattern or printing applied thereto, which may include one or more shapes. Accordingly, in various exemplary embodiments, the first visual characteristic may be a color and the second visual characteristic may be a different color, with one or both of the colors applied to the wrapper and/or fastening element. In another embodiment, the first visual characteristic may be a color, and the second visual characteristic may be an embossment, pattern, shape (e.g., fastening element) or side seal. In yet another embodiment, the first visual characteristic may be a first embossment, printing or dying pattern and the second visual characteristic may be a second embossment, printing or dying pattern.

Referring to FIG. 4, the bulk packaging component 76 also has at least a first and second visual characteristic. For example, the outer surface 80 of packaging component may have one or more colors, printings, side seal configurations, etc. In a desired embodiment, the first and second visual characteristics of the bulk packaging component are correspond to and are coordinated with the first and second visual characteristics of the individual packaging component.

Referring to FIGS. 3, 5 and 6, the product component 10 also has at least a first and second visual characteristic that correspond to and are coordinated with the first and second visual characteristics of one or both of packaging components 50, 76. For example, a portion of one or more of the outer cover 46, peel strip 28, absorbent core 48, surge layer 4 or tissue layer 6 has a first visual characteristic, while another portion thereof, or another element, has a second visual characteristic, with the first and second visual characteristics corresponding to and being coordinated with the first and second visual characteristics of one or both of the packaging components. In various embodiments, portions of the absorbent core, surge layer and/or tissue layer, or any other substrate disposed between the liner and outer cover, are visible through the liner, which is at least partially see-through, while in other layers such interior components may not be visible. In addition, a body-side surface of the outer cover may be visible through the liner and/or absorbent core on the body-side surface of the absorbent product.

In one embodiment, the outer cover 46 and the wrapper 52 are the same first color, while the outer surface of the peel strip 28 and the fastening element 72 are the same second color, meaning the colors have similar hues, saturation and/or luminosity, or combinations thereof, as defined below. At the same time, the first and second colors of the product component are different from each other, meaning that the colors have a different hue, saturation and/or luminosity, or combinations thereof. In another embodiment, the outer cover 46 and the fastening element 72 are the same first color, while the outer surface of the peel strip 28 and the wrapper 52 are the same color. The various colors include without limitation white, black, yellow, orange, purple, green, red, blue, and pink. In other embodiments, one of the core 48, surge layer 4 and tissue layer 6 are a first color, preferably non-white, which is visible to the user through the liner 44 and which corresponds to a first color of the wrapper 52, product component 76 and/or fastener element 72. For example, the outer cover can be configured as a purple, blue, pink or green, with the absorbent core being white or some other different color, and with one or both of the surge layer and tissue layer also being purple, blue, pink or green. At the same time, one of the wrapper and fastening element are made purple, blue, pink or green, with the other of the wrapper and fastening element being white or the other different color. In other embodiments, one of the product and/or packaging components can have a color gradient, wherein the color transitions from one color to another, or one or more of the properties thereof changes over a distance.

In other embodiments, the liner, peel strip or outer cover are provided with an embossment (i.e., macro-embossing that is discernable to the user) or a printing or dying pattern that corresponds to an embossment or printing pattern on the wrapper, fastening element or packaging component.

Of course, it should be understood that the packaging components 52, 76 and product components 10 could have more than two coordinated visual characteristics, including for example three characteristics such as a pair of colors and an embossment and/or printing/dying, or three colors, or any combination of the visual characteristics set forth above or otherwise known in the art.

As explained above, in one embodiment, the first and second visual characteristics are configured as first and second colors. Desirably, each of the first and second colors are different, meaning the colors have a different hue. One or more colors may also be different by virtue of having a different luminosity and/or saturation/vividness. Saturation/vividness is the intensity of the color from pale to dark. The elements may also have a different gloss/finish, from a matte finish, which tends to diffuse or scatter light, to a gloss finish, which is specular.

Referring to FIG. 13, hue is measured by the angular position around the circle 110. Two colors are considered different if they have first and second hues that are more than ±0.50 degrees from each other on the circle 110, alternatively ±5 degrees, alternatively ±30 degrees, alternatively ±90 degrees, alternatively ±150 degrees and alternatively ±175 degrees. Value (luminosity) is measured along the Z-axis 112 between white and black. Colors are considered different if they have a value difference of at least 1% of maximum (Polaroid white reference standard). A value of one equates to white, while a value of 0 equates to black. Saturation/vividness is measured along the length of the radius (r). Colors are considered different if they have a saturation difference of at least 2.5% of maximum.

At the same time, the first color of the personal care product component, such as the absorbent product, can be coordinated with the first color of one or both of the packaging components. In one embodiment, the second color of the personal care product component, such as the absorbent product, is also coordinated with the second color of one or both of the packaging components. The coordination of the colors is most desirable at distances of less than two feet, such that it is visible to the user of the product. At the same time, the coordination can provide a disguising aspect that is effective for an observer who is greater than 2 to 3 feet away from the product.

Two colors are considered coordinated if they have first and second hues that are within about ±120 degrees of each other on the circle 13, alternatively within ±30 degrees, alternatively within ±15 degrees, alternatively within ±10 degrees, alternatively within ±5 degrees of each other, or alternatively within ±50 degrees of each other. Colors are also considered coordinated if they have a value (luminosity) difference of less than about 5% of maximum, alternatively less than about 30% of maximum or alternatively less than about 1% of maximum. Colors are also considered coordinated if they have a saturation difference of less than about 5% of maximum, alternatively less than about 30% of maximum or alternatively less than about 2.5% of maximum.

The hue, luminosity and saturation/vividness are measured as follows using the following equipment calibrated in the following way.

Equipment

Quantitative colorimetric measurements are typically made using a colorimeter or spectrophotometer. However, these instruments typically have large apertures (˜1 cm) requiring a large color block for meaningful determination, making them unsuitable for color determination of graphics that may be composed of narrow lines or points whose width is much less than the instrument aperture. Therefore, a Zeiss KS400 Image Analysis system was used for feature identification and calorimetric measurement.

The Zeiss KS400 used a Zeiss AxioCam color CCD camera (1300×1030 pixels, 3 channel color, 8 bit per channel) equipped with a 20 mm AF-Nikkor lens (f/2.8). The camera was mounted vertically facing down onto a sample stage and had an effective field of view was 97×80 mm. Incident sample stage illumination was by four incandescent floodlamps (Sylvania) on a double Variac (70%; 90%), resulting in an illuminance of approximately 11,000 lux. The lamps were above the left and right edges of the sample stage directed towards the field of view at approximately 45 degrees.

Calibration

The camera black reference was with the lens cap on. The camera white reference was a Polaroid 803 positive with 15 ms exposure. To account for the warm color illumination bias of the floodlamps, the red, green, and blue (RGB) values were offset using the white selection tool in the KS400 software, resulting in corrected RGB values that yielded a white image.

Sample Setup and Image Acquisition

Samples are placed on the stage (normal viewing angle) and under ¼″ plate glass to minimize topographical effects. Images of the color-bearing graphical portion are acquired at 15 ms exposure.

Image Analysis

Image analysis is performed in Matlab (v.6.5.1, release 13; Mathworks, Inc) with the Image Processing Toolbox (v4.0). RGB images were converted to hue, saturation, and value (HSV) space using Matlab's hsv2rgb.m command. Choosing a saturation lower limit of 0.05 (0-1 scale) resulted in practical detection of all the colored/inked portions of the graphic. The hue, saturation, and value (i.e. luminosity) densiometric distributions were calculated for the detected regions in each image.

In one embodiment, and referring to FIGS. 3 and 5, the packaging component 50 is made of a combination of blue fibers. The blue of the fibers has a consistent hue but the process of adding the blue to the fibers creates blue fibers of varying saturation. So, the fibers appear to vary in vividness with some of the fibers almost appearing white and the pouch appearing as a random assortment of blues. This random assortment of blues allow the packaging component 50 to coordinate to the various colors in the product component 10 elements, for example a white peel strip 28 and blue baffle 46. For example, in one embodiment, the difference in the hue between the outer surface of the baffle, which is blue, and the pouch, which is blue, is 13.6 degrees and the difference in the value (luminosity) is 0.6% of maximum.

This coordination also allows the packaging component to hide or obscure the product component disposed therein. In essence, the coordination leads the observer (standing 2 or more feet away—as opposed to the user standing within 2 feet of the product) to consider the packaging and product components as a single unit, rather than as two separate units. Therefore, the observer has a more difficult time distinguishing the pad within the pouch. In this way, the coordination serves a dual purpose of coordinating the product for the user (i.e., providing a more aesthetically appealing product) while hiding the product from observers.

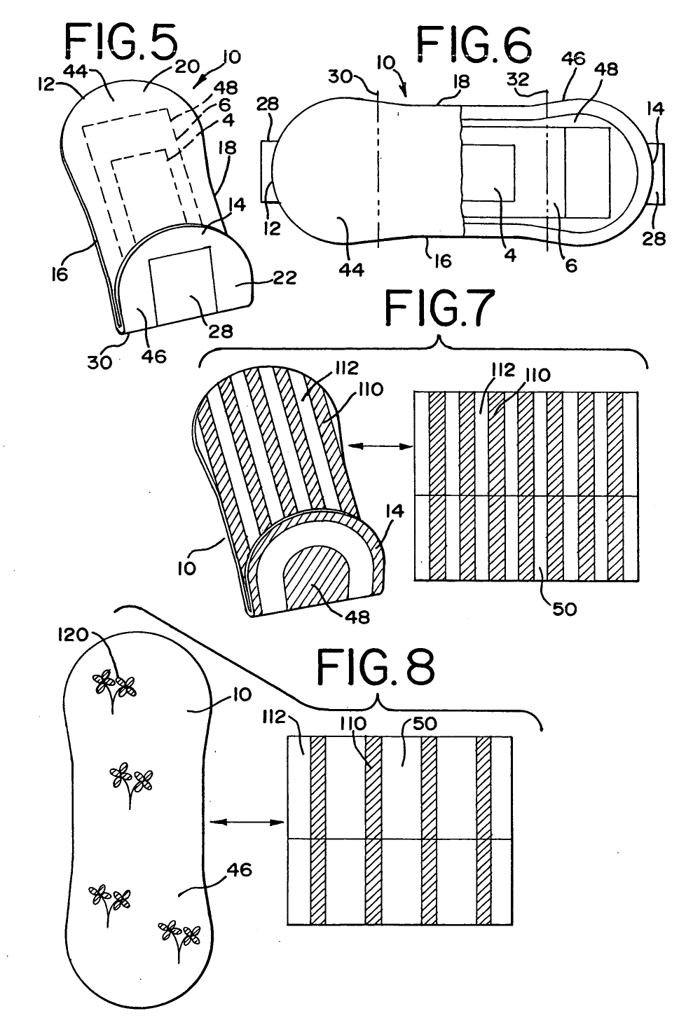

Referring to FIGS. 7-12, different embodiments of various product and packaging components are shown with various coordinating visual characteristics. For example, as shown in FIG. 7, the absorbent product 50 is shown as having a pattern of alternating strips 110, 112 of first and second colors. Likewise, the packaging components 50, 118, each have the same pattern in the same colors. It should be understood that two patterns are coordinated if they both have at least one substantially identical element, or if the overall distribution of elements in a certain region is substantially identical. Therefore, and for example, the patterns of the product and packaging components in FIG. 7 are coordinated in two ways, (1) each component has a substantially identical element (e.g., one of the stripes 110, 112) and (2) both components have a substantially identical distribution of elements 110, 112 thereover, i.e., a plurality of similarly dimensioned stripes alternating in color. For example, a checker board pattern would be coordinated by the shape of the individual elements (squares) as well as by the overall distribution thereof (alternating colors and positioning). It should be understood that for the first type of pattern coordination (substantially identical elements), the elements do not have to be of the same size or oriented in the same orientation, but rather that they merely have substantially the same shape. With the respect to the second type of pattern coordination, the individual elements do not even have to have the same shape, as long as the overall distribution is coordinated. It should be understood that the product and packaging components 10, 50, 118 of FIG. 7 are also coordinated by way of the colors of the stripes, which have one or more coordinated hues, luminosities and saturation/vividness values.

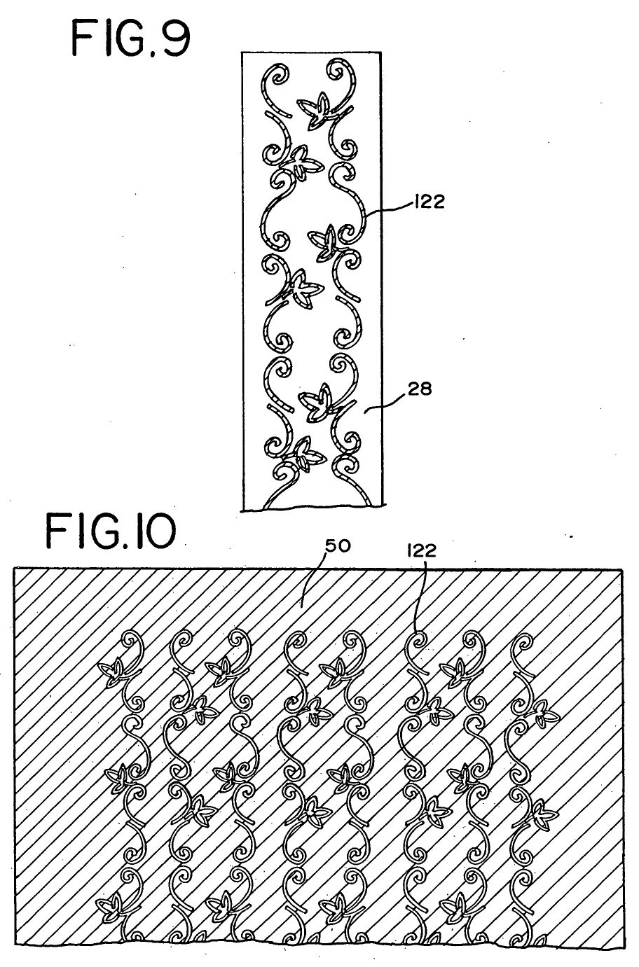

Referring to FIG. 8, the product component 50 is coordinated with the first packaging component 50 first by way of the colors of the pattern element 120 and the pattern element 110 and second by way of the color of the backsheet 46 and the pattern element 112. The product component 50 is coordinated with the second packaging component 118 first by way of the pattern elements 120, second by way of the color of the pattern elements 120, and third by way of the color of the backsheet 46 and color of the base sheet 140.

Referring to FIGS. 9 and 10, a peel strip 28 is configured with a pattern 122 of ivy, while the packaging component 10 is configured with a coordinated pattern 122 of ivy. The patterns 122 are coordinated, i.e., substantially the same shape, even thought they are of different sizes. The peel strip 28 is further coordinated with the packaging component 10 by way of the color of the pattern 122 on the peel strip with the color of the background on the packaging component, which are both blue for example with coordinated colors, including for example hues, saturations and/or luminosities, or combinations thereof. Likewise, the color of the background of the peel strip 28 is coordinated with the color of the ivy pattern 122 on the packaging component.

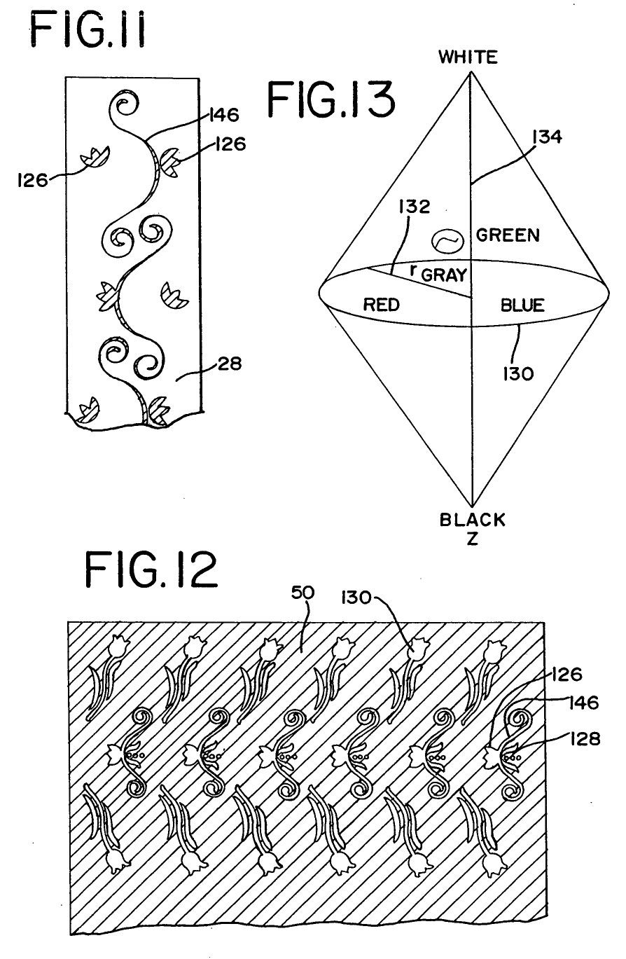

Referring to FIGS. 11 and 12, a peel strip 28 is configured with a pattern 126, 146 of tulips and scrolls, while the packaging component 10 is configured with a coordinated pattern 126, 146 of tulips and scrolls. The patterns 126, 146 are coordinated, i.e., substantially the same shape, even though they are of different sizes and notwithstanding that additional pattern elements 128, 130 are also present on the packaging component. The peel strip 28 is further coordinated with the packaging component 10 by way of the color of the patterns 126, 146 on the peel strip with the color of the background on the packaging component, which are both blue for example with coordinated colors, including for example hues, saturations and/or luminosities, or combinations thereof. Likewise, the color of the background of the peel strip 28 is coordinated with the color of the patterns 126, 146, 128 and 130 on the packaging component.

In some embodiments, the product and packaging components can also be coordinated by other sensory characteristics, for example touch and smell. For example, the packaging component can have the same texture as the product component, so as to provide a coordinated tactile characteristic. In some embodiments, the tactile characteristic is formed by an embossment, or different embossments. Similarly, separate pieces of the same type of material may be applied to the product and packaging components.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A visually coordinated absorbent product comprising:
a product component comprising a body side liner, a garment side outer cover and an absorbent core disposed between said body side liner and said garment side outer cover, wherein said product component has at least a first and second visual characteristic, wherein said first visual characteristic is different than said second visual characteristic; and
a packaging component having at least said first and second visual characteristics, wherein said product component is disposed in said packaging component.

2. The visually coordinated absorbent product of claim 1 wherein said product component comprises a peel strip removably connected to said garment side outer cover, wherein at least a portion of said garment side outer cover is configured with said first visual characteristic and at least a portion of said peel strip is configured with said second visual characteristic.

3. The visually coordinated absorbent product of claim 2 wherein said product component further comprises a substrate disposed between said body side liner and said absorbent core, wherein said substrate has one of said first and second visual characteristics.

4. The visually coordinated absorbent product of claim 3 wherein said substrate comprises at least one of a tissue layer and a surge layer.

5. The visually coordinated absorbent product system of claim 1 wherein said first and second visual characteristics comprise first and second colors.

6. The visually coordinated absorbent product of claim 5 wherein said first color is white and wherein said second color is selected from the group consisting of black, yellow, orange, purple, green, red, blue and pink.

7. The visually coordinated absorbent product of claim 5 wherein said first color of said product component has a first hue and wherein said first color of said packaging component has a second hue, wherein said first hue is within about 120° of said second hue.

8. The visually coordinated absorbent product of claim 7 wherein said second color of said product component has a third hue and wherein said second color of said packaging component has a fourth hue, wherein said third hue is within about 120° of said fourth hue.

9. The visually coordinated absorbent product of claim 7 wherein said first color of said product component has a first value and wherein said first color of said packaging component has a second value, wherein the difference between said first and second values is within 1% of maximum.

10. The visually coordinated absorbent product of claim 7 wherein said first color of said product component has a first saturation and wherein said first color of said packaging component has a second saturation, wherein the difference between said first and second saturations is within 2.5% of maximum.

11. The visually coordinated absorbent product of claim 1 wherein at least one of said first and second visual characteristics comprises an embossment.

12. The visually coordinated absorbent product of claim 1 wherein at least one of said first and second visual characteristics comprises a pattern.

13. The visually coordinated absorbent product of claim 1 wherein said packaging component is sized and configured to hold a single, individual product component.

14. The visually coordinated absorbent product of claim 13 wherein said packaging component comprises a wrapper element having said first visual characteristic and a fastening element having said second visual characteristic.

15. The visually coordinated absorbent product of claim 1 wherein said packaging component is sized and configured to hold a plurality of said product components.

16. The visually coordinated absorbent product of claim 1 wherein said packaging component comprises a non-woven material.

17. The visually coordinated absorbent product of claim 16 wherein said non-woven material has a basis weight of between about less than about 1.0 osy.

18. A visually coordinated absorbent product comprising:
a product component comprising a body side liner, a garment side outer cover and an absorbent core disposed between said body side liner and said garment side outer cover;
a first packaging component having at least first and second visual characteristics, wherein said product component is disposed in said first packaging component; and
a second packaging component having said at least said first and second visual characteristics, wherein said first packaging component is disposed in said second packaging component.

19. The visually coordinated absorbent product of claim 18 wherein said product component comprises said at least said first and second visual characteristic.

20. The visually coordinated absorbent product of claim 18 wherein said first and second visual characteristics comprise first and second colors.

21. The visually coordinated absorbent product of claim 18 wherein at least one of said first and second visual characteristics comprises a pattern.

22. The visually coordinated absorbent product of claim 18 wherein said first packaging component comprises a wrapper element having said first visual characteristic and a fastening element having said second visual characteristic.
